(19) United States
(12) Patent Application Publication
(10) Pub. No.: US 2011/0130809 A1
(43) Pub. Date: Jun. 2, 2011

(54) Pacing and Stimulation Apparatus and Methods

(75) Inventor: Mark Zdeblick, Portola Valley, CA (US)

(73) Assignee: PROTEUS BIOMEDICAL, INC., Redwood City, CA (US)

(21) Appl. No.: 12/600,586

(22) PCT Filed: Nov. 13, 2009
(86) PCT No.: PCT/US09/64487

§ 371 Date: May 25, 2010

Related U.S. Application Data
(60) Provisional application No. 61/114,441, filed on Nov. 13, 2008.

(51) Int. Cl. A61N 1/36

(52) U.S. Cl. 607/62

(57) ABSTRACT

System, device and method of variable pacing and stimulation are provided. In one example device, one or more lead integrated circuits may programmably control one or more variability parameters. One or more electrodes may provide electrical stimulation. Each electrode may be individually addressable by at least one of the lead integrated circuits and controlled by at least one variability parameter. Electrodes may be intentionally shorted together during time intervals that fall between times of stimulus, so as to dissipate potentials that might otherwise persist and cause electrode degradation.

### CROSS-REFERENCE TO RELATED APPLICATIONS

This application claims the benefit of U.S. application No. 61/114,441, filed Nov. 13, 2008, which application is incorporated by reference for all purposes.

### BACKGROUND

The present invention relates generally to medical therapy systems, devices, and methods. More specifically, the invention relates to systems, devices, and methods for pacing and stimulation.

Various defects and conditions may adversely affect various systems of the body. These systems include, for example, the circulatory system; the digestive system; the endocrine system; the immune system; the integumentary system; the lymphatic system; the activity system; the nervous system; the reproductive system; the respiratory system; and the urinary system.

By way of illustration, circulatory system conditions such as cardiac-related defects may lead to congestive heart failure (CHF), fatal cardiac arrhythmia, etc. Conditions related to the nervous system may result in chronic and/or acute pain sensations. Common chronic pain complaints include headache; low back pain; cancer pain; arthritis pain; neurogenic pain; i.e., pain resulting from damage to the peripheral nerves or to the central nervous system; and psychogenic pain, i.e., pain not caused by past disease, injury, or any visible sign of damage inside or outside the nervous system. Urinary system defects include lack of voluntary control of excretory functions, incontinence or urge, etc.

Various devices may be used to provide therapies for such conditions. For example, stimulation devices may be used to facilitate electrical stimulation and/or pacing of a heart to treat defects in the heart's conduction system. Such devices may rely on fixed processes, sequences, programs or the like to deliver such therapies. For example, a pacing device such as a biventricular pacing device may provide a fixed pattern of electric pulses having a particular timing, duration, amplitude, frequency, etc.

Therapies incorporating such fixed parameters may not be suitable for optimal treatment and management of various defects and conditions. Although a patient may be responsive initially to such therapies, a decrease in responsiveness resulting from repeated exposure to a stimulus, i.e., habituation, may occur. Habituation may eventually render a therapy ineffective.

Moreover, once the therapy becomes ineffective, devices used to deliver such therapies may need to be removed and replaced, resulting in surgical procedures, patient trauma, extended recovery times, etc. In addition, any or all of the effects of removal and replacement of devices may be performed at risk to the patient; may exacerbate the underlying condition or defect; and may further hinder the patient's treatment and progress.

Further, such therapies may deliver electrical stimulus to areas of tissues not intentionally targeted causing untoward results, e.g., tissue overstimulation, disruption of rhythm, stimulation of areas resulting in pain responses, etc.

Therefore, it would be desirable to have systems, devices, and methods for optimizing such medical therapies and enhancing patient treatment results, where such therapies have continued and prolonged beneficial results.

### BRIEF DESCRIPTION OF THE FIGURES

FIG. 1 illustrates an exemplary placement of a variable pacing and stimulation device relative to a portion of the spinal column of the nervous system of a human subject.

FIG. 2 illustrates an exemplary lead portion of the variable pacing and stimulation device of FIG. 1 relative to a portion of the spinal column of the nervous system of a human subject.

FIG. 3 illustrates an exemplary segmented electrode satellite of the lead portion of FIG. 2.

FIG. 4 illustrates an exemplary array configuration of the variable pacing and stimulation device.

FIG. 5 illustrates first unshielded, bipolar, biphasic pacing results.

FIG. 6 illustrates second unshielded, bipolar, biphasic pacing results.

FIG. 7 illustrates first shielded, bipolar, biphasic pacing results associated with variable pacing stimulation devices, systems, and methods.

FIG. 8 shows a prior-art way of driving electrodes, using blocking capacitors.

FIG. 9 shows a first embodiment of the “shorting” approach.

FIG. 10 shows a second embodiment of the “shorting” approach.

FIG. 11 shows the satellite of FIG. 3 in schematic portrayal.

FIG. 12 shows the chip 403 of the satellite of FIG. 11 in schematic detail.

FIG. 13 shows a third embodiment of the “shorting” approach.

### DETAILED DESCRIPTION

Generally, various aspects of the variable pacing and stimulation invention provide pacing and/or stimulation therapies for various health conditions, wherein such therapies incorporate programmably-variable parameters, algorithms, features, etc. (hereinafter, “parameters”). The variable parameters may facilitate, inter alia, focusing stimulation and/or pacing fields, modulating intensity of energy in a focused field, automatically modulating to mitigate habituation, and automatically adjusting to other sensed parameters such as activity or sleep, etc. The variable parameters may optimize various therapies and may avoid, eliminate, or mitigate various adverse consequences.

Potential clinical value of modulating or varying pacing and stimulation parameters may span multiple applications, including cardiac, pain, movement disorders, incontinence, gastro-intestinal motility disorders, hypertension, and sleep apnea. Various aspects may be implemented in wired or wireless form factors, e.g., with dual electrodes, multi-electrode arrays, arrays of multi-electrode arrays, etc. Generally, the invention includes devices, systems, and methods for controlling one or more modular circuits, e.g., lead integrated circuits (lead ICs), associated with one or more electrodes, including the functionality necessary to provide variability parameters and combinations thereof.

Certain aspects may be directed to in-body therapies and may include, for example, implantable medical devices. The term “implantable medical device”, as used herein, refers to a device configured to be positioned at least partially on a living body, at least partially in a living body, or a combination thereof. For example, the implantable medical device may include a lead having various electrode configurations communicably associated with controller circuitry, a power source, etc. More particularly and illustratively, the implantable medical device may comprise one or more leads with multiple in-line segmented electrode satellites, wherein each electrode is independently controllable, as well as power/data wire(s) for multiplexing the multiple segmented electrode satellites. Various configurations of devices which may be used conjunction with this invention are described/disclosed in PCT application no. PCT/US2003/039524 published as WO 2004/052182; PCT application no. PCT/US2005/031559 published as WO 2006/029090; PCT application no. PCT/US2005/046811 published as WO 2006/069322; PCT application no. PCT/US2005/046815 published as WO 2006/069323; PCT application no. PCT/US2006/048944 published as WO 2007/075974; and U.S. application Ser. No. 11/939,524 published as US 2008-0114230 A1. Each of the aforementioned applications is herein incorporated by reference in its entirety.

It will be appreciated that the aforementioned configuration is for illustrative purposes only and that various other components and configurations are possible.

FIG. 1 illustrates an exemplary placement of a variable pacing and stimulation device 100 relative to a portion of the spinal column 102 of the nervous system of a human subject. The variable pacing and stimulation device 100 may include variable numbers of electrode(s) in various configurations, e.g., segmented electrode satellites 104, and may be in communication, e.g., wireless or wired electrical communication, with various components. Such components may include, for example, network connector 106 to communicably connect the segmented electrode satellites 104 to, for example, a power source 108 such as an implantable, rechargeable battery via a lead connection 110 such as a single lead connection.

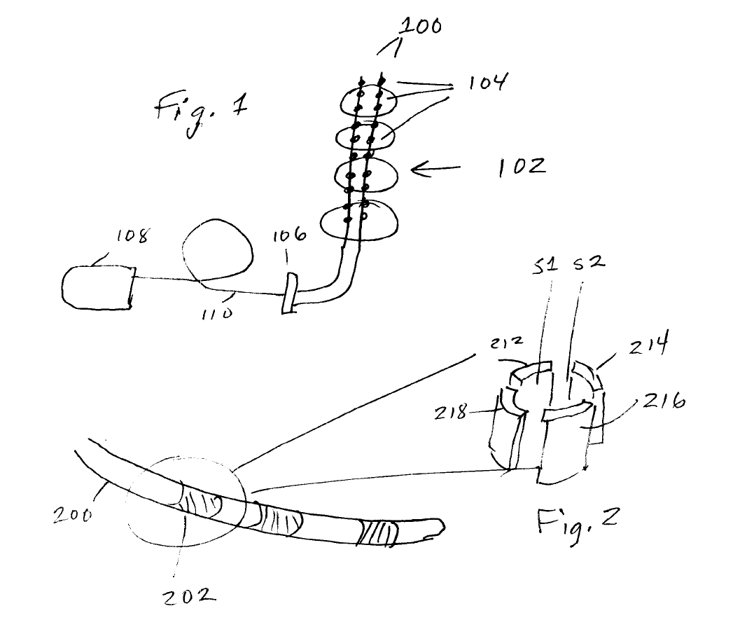

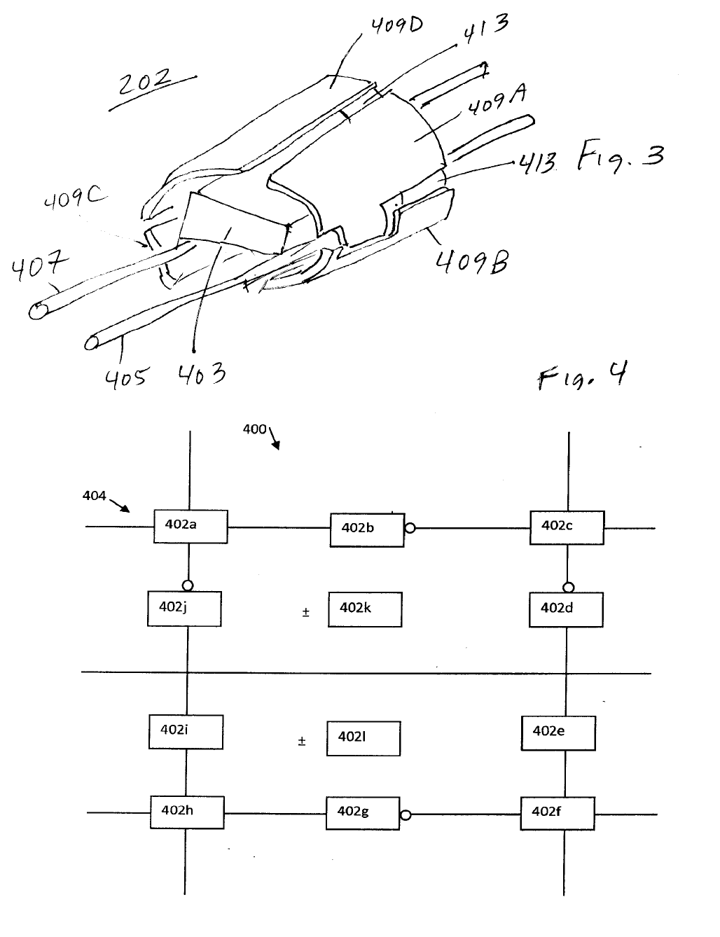

FIG. 2 illustrates an exemplary lead 200 portion of the variable pacing and stimulation device 100 of FIG. 1 relative to a portion of the spinal column 100 of the nervous system of a human subject. The lead 200 may comprise, for example, one or more segmented electrodes 202 having multiple segments, e.g., four-segmented electrodes 212, 214, 216 and 218, respectively. Other segment configurations are possible, e.g., six-segmented electrodes, etc. In various aspects, each segmented electrode 202 is individually controlled, i.e., independently of the other segmented electrodes 202, and directly controllable by an IC to which the segmented electrode 202 is directly connected (as shown in FIG. 3). Communication vehicles such as power/data wires S1 and S2, facilitate overall power and data communication to/from/within the lead 200, e.g., to and from a power source, controller circuitry, etc.

FIG. 3 illustrates an exemplary segmented electrode satellite 202 of the lead 200 portion of FIG. 2. The segmented electrode satellite 202 may include, for example, multiple segments, e.g., two, three, four, etc. The segmented electrode satellite 202 will include connections between IC 403 and elongated conductive members 405 and 407.

IC 403 is attached to quadrant electrodes 409A, 409B, 409C and 409D. Quadrant electrodes 409A, 409B, 409C and 409D are joined together with PEEK material 413.

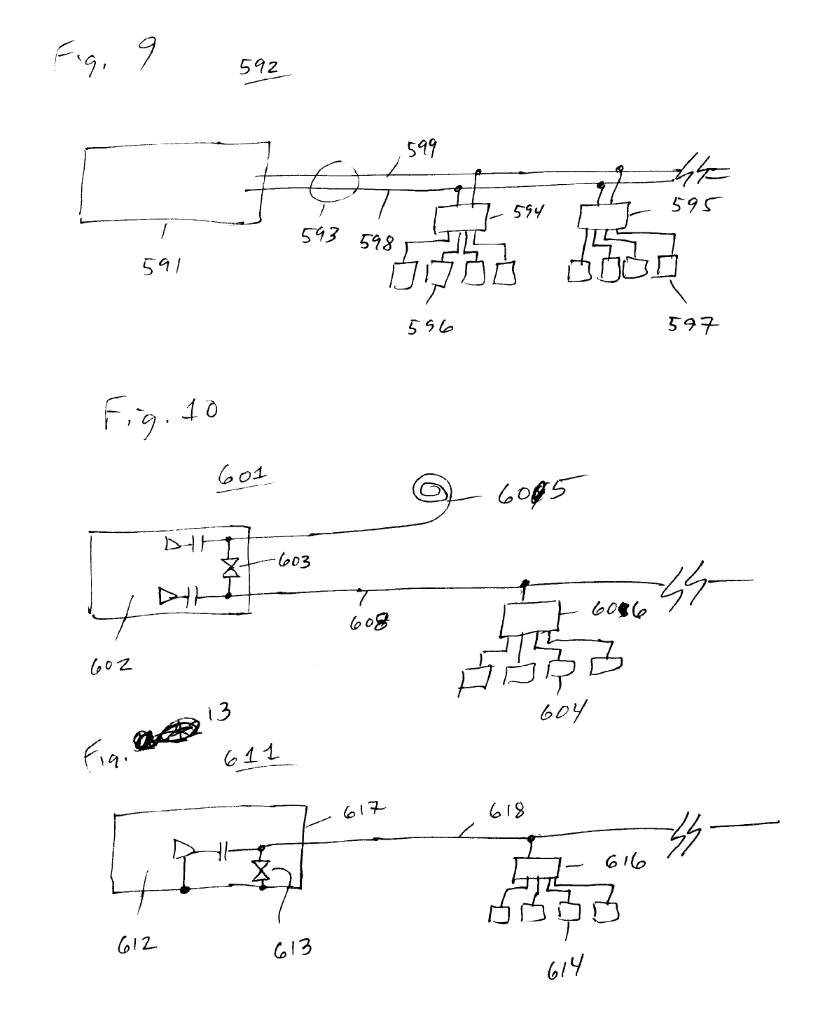

FIG. 11 shows the satellite of FIG. 3 in schematic portrayal. One or more satellites 202 are distributed along the length of the lead 200 (FIG. 2). Each satellite has a chip 403 which derives power and receives control signals from conductors S1 and S2.

FIG. 12 shows chip 403 in schematic detail. Block 455 extracts power from S1 and S2 and provides power to other blocks. Block 454 derives clock and data from S1 and S2 and passes data to core 453 which provides computational functions much like a microcontroller. Core 453 in turn controls switching fabric 452 which selectively connects one or another of lines 451 to lines S1 and S2. Lines 451 in turn connect to electrodes 409A, 409B, 409C, 409D.

Switching fabric 452 can in turn optionally include circuitry that modulates stimulation signals on lines S1 and S2, as discussed below in more detail. For example line S1 might offer some DC potential relative to line S2. Line S2 might be connected to electrodes which thus define a ground or neutral potential, or which define a shielding potential. In such a case, the switching fabric 452 could connect line S1 to a particular stimulus electrode in any of particular ways. One way of connecting would be a pulse-width-modulated connection providing an approximated sine wave to the stimulus electrode. Or the way of connecting might be a square wave of any of several different frequencies. Such modulations may be accomplished with simple on-off switches. Or a spread-spectrum modulation (a sequence-type modulation) may be employed, or any of several other modulations that use switching semiconductors capable of being driven with intermediate positions between “on” and “off”.

The lead IC 403 may provide a basic cross-connect functionality between bus wires, e.g., two bus wires, and electrodes, e.g., one or more electrodes, multiple electrodes in a satellite configuration, multiple individual electrodes, etc., that interact with the body tissue.

The cross-functionality includes at least one variability parameter selected from a group consisting essentially of a voltage variability parameter; spread spectrum variability parameter, wherein the signal may be deliberately spread in the frequency domain, resulting in a signal with a wider bandwidth; a pacing variability parameter, wherein the pulses are delivered at varied paces; a delay variability parameter, wherein the delay between the right-sided pulse and the left-sided pulse is varied in time; a frequency variability parameter, wherein the signal is varied in frequency; an interval variability parameter, wherein the intervals between signals are varied; an amplitude variability parameter, wherein the amplitude of the signal is varied; a component variability parameter, wherein various components are configured; a state variability parameter, wherein an “on” state and an “off” state are varied; a blocking variability parameter, wherein a predetermined and/or portion of electrodes are blocked; a potential variability parameter, wherein the voltage potential is varied; a focus variability parameter, etc. In this manner, stimulation sites may be targeted via the one or more of different electrodes while providing programmable treatment variations associated with the stimulation to avoid, eliminate, or reduce an habituation response.

In various aspects, the variability parameter may be generated by a random generation scheme, a pseudo-random generation scheme, or determined by various other schemes, programs, etc.

More particularly, in certain aspects, the voltage variability parameter includes the functionality necessary to convert a pacing pulse from a voltage that is present across the two bus wires, e.g., S1 and S2, to a different, programmable voltage.

The voltage variability parameter comprises, for example, differing the amplitude of the voltage from that across the wires; differing the timing of the pulse to be delivered to the tissue; or a combination of both. In certain aspects, the amplitude and timing may be controlled at the lead IC level. One configuration, for example, includes a neural stimulation lead having multiple, e.g., sixteen, satellites, each satellite having four electrodes arranged circumferentially around its perimeter, all of these connected by two wires to an Implantable Pulse Generator (IPG). In various aspects, the variability parameter may be generated by a random generation scheme. The random generation scheme comprises, for example, using one of the published methods for generating a random number using discrete logic. The variability parameter may be generated by a pseudo-random generation scheme. The pseudo-random generation scheme comprises, for example, using one of the published methods for generating a pseudo-random number using discrete logic.

Various other generation schemes may also be used.

To illustrate an application of the foregoing, pain therapy may be facilitated by providing various stimulation currents to the body from different electrodes at the same time. Consider a case where there are four satellites M0, M1, M2, and M3 along the length of lead 200 (FIG. 2). For example, satellites M0 and M1 could be programmed to provide a 1V, 100 Hz stimulation field while satellites M2 and M3 are providing a 2V, 1000 Hz stimulation field. Variability parameters describing when pacing pulses are to be fired, the pulse width of the pacing pulse, and the amplitude of the pacing may be stored on the lead IC. The circuitry on the lead IC may convert the voltage that appears on S1 and S2 into the programmed voltage and deliver it, i.e., a blocking variability parameter, to a portion of selected electrodes. In this manner, various therapies may be enhanced while mitigating habituation.

One example application includes cardiac pacing therapy where the pacing rate varies according to the patient's activity rate, e.g., sedentary versus extreme activity. Based on the varying pacing rates, determination of patient activity may be derived or ascertained via various devices and related data communicated via a variety of means to the circuitry, e.g., wired communication or wireless communication.

Another example application includes pain therapy, where one or more electrodes are located in the epidural region of the spinal column and where the stimulation is controlled via various electrodes of the total electrodes and via various stimulation parameters to stimulate certain tissue regions, e.g., midline dorsal column fibers of the spinal column, as a means for masking/blocking/mitigating pain, while avoiding stimulation of other tissue regions, e.g., lateral fibers and dorsal roots, which if stimulated may have adverse results such as acute pain, increased pain, etc.

To further illustrate, and in various aspects, one or more electrodes may be at selectively variable states, e.g., on or off (active or non-active), etc. The lead IC may control operation of the electrodes to determine a proper, e.g., desired, state. In one example configuration, the electrodes may be turned off via various means, e.g., an external wireless remote control in communication with the IC lead. In this manner, better control may be exercised when employing electrodes and other stimulus devices during exposure to environments/procedures traditionally incompatible with procedures such as magnetic resonance imaging (MRI), etc. In certain aspects, the focus variability parameter includes the functionality necessary to focus and/or refocus a stimulation field toward certain target sites and away from other target sites. Various configurations include, for example, the use of various voltages on arrays of electrodes. An array of electrodes may vary in size, e.g., two rows, four rows, twenty rows, thirty rows, etc. Discrete examples include, for example, a 2×10 array, a 4×4 array, etc. One or more arrays of arrays are also contemplated, e.g., four 2× arrays, etc.

FIG. 4 illustrates an exemplary array configuration 400 of the variable pacing and stimulation device having multiple electrodes 402. A portion of the electrodes 402, e.g., electrodes 402a-402j, are configured to surround the remaining electrodes 402, e.g., electrodes 402k, 4021. For ease of reference, the portion of electrodes 402 surrounding the remaining electrodes 402 is sometimes referred to herein as a “ring 404” of electrodes. It will be appreciated that the surrounding electrodes may be configured according to various topologies. The ring 404 of electrodes, e.g., electrodes 402a-402j, may be programmed to be at a neutral voltage. Inside the ring 404 of electrodes, the remaining electrodes, e.g., electrodes 402k and 4021, may be programmed to alternate between voltages that are positive or negative with respect to the neutral voltage, i.e., a voltage variability parameter.

For example, a 20 kHz 5V AC voltage is placed through a blocking capacitor on S1 and S2. This is converted on the lead IC to DC 5V and 0V sources. In addition, the circuitry on the lead IC also converts the voltage to a 2.5V source, which is connected to the ring of electrodes programmed to be the “neutral ring”. A counter on the lead IC is set up based upon the 10 kHz signal appearing on the bus. Timing, amplitude, duty cycle and active-electrode-location parameters are stored on the lead IC. At an appropriate time (as determined by the counter on the lead IC), the 0 V source is connected to one or more electrodes, and the 5V source is connected to other electrodes. The electrodes stay connected for about 1 ms (or whatever parameter was stored to determine stimulation duration) and are then disconnected and then reconnected to the opposite voltage sources for exactly the same period of time (as determined by the counter). To achieve charge balance on the electrodes, the electrodes are then both connected to the 2.5V voltage source. In this manner, the stimulation may be focused on a target site and refocused elsewhere multiple times, as desired. In one example, the stimulation may be focused on treatment-responsive areas, e.g., in a nervous system application, the midline dorsal column fibers of the spinal column, and may be focused away from areas which may have adverse results if stimulated, e.g., in the nervous system application, lateral fibers and dorsal roots.

To illustrate the foregoing concepts, various finite element models were tested. First, two models without the afore-described ring of electrodes, i.e., “unshielded” were modeled using a first unshielded, bipolar, biphasic pacing model and a second unshielded, bipolar, biphasic pacing model. Next, four models having the afore-described ring of electrodes, i.e., “shielded” were tested: a first shielded, bipolar, biphasic pacing model; a second shielded, bipolar, biphasic pacing model; a first shielded, 3-state, biphasic pacing model; and a second shielded, 3-state, biphasic pacing model. The results are visually illustrated in FIGS. 5-10.

FIG. 5 illustrates a first unshielded, bipolar, biphasic pacing modeled result. When 0 V is applied on upper electrode 402k, and when 5V is applied on the lower electrode 4021, tissue relatively far away from the tissue site of the upper electrode and the lower electrode is affected, e.g., tissue affected by 2.44 V shown in the upper half of the diagram (area 471) and tissue affected by 2.56 V shown on the lower half of the diagram (area 472). From this, it may be concluded that application of electrical stimulus to tissue sites of the upper electrode and the lower electrode result in electrical stimulus to a relatively large area of tissue not associated with the tissue sites of the electrodes, i.e., a relatively large area of tissue in which electrical stimulus is preferably to be avoided. Note that similar results would be obtained by using currents instead of “voltage” in this example.

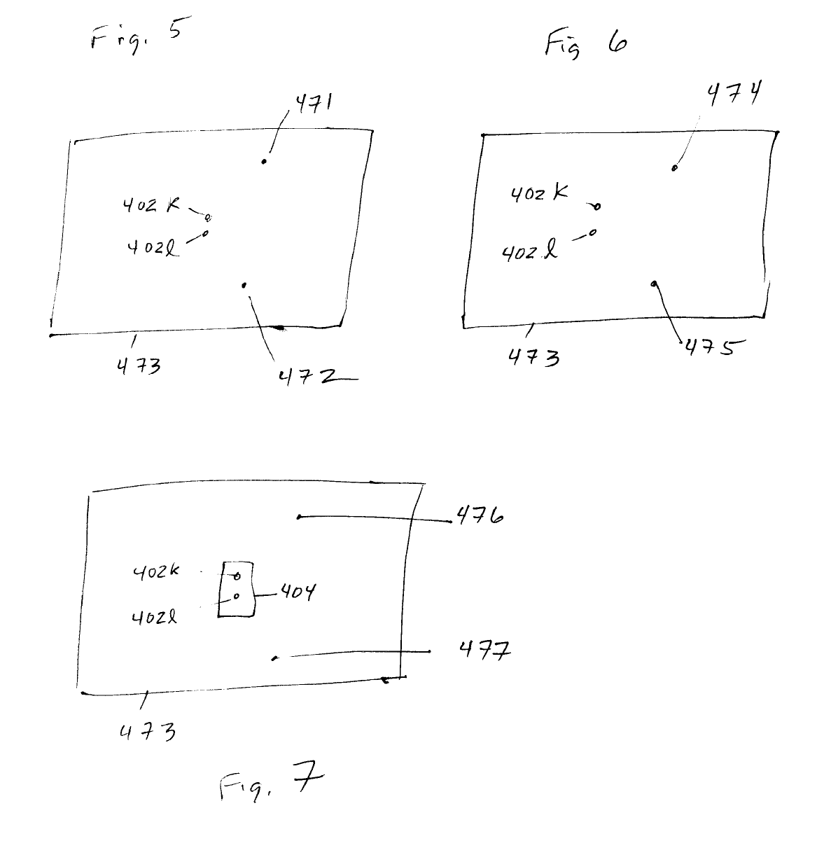

FIG. 6 illustrates a second unshielded, bipolar, biphasic pacing modeled result. As can be seen, when 0 voltage is applied on upper electrode 402k, and when −5 V is applied on the lower electrode 4021, tissue relatively far away from the tissue site of the upper electrode and the lower electrode is affected, e.g., tissue affected by −2.44 V shown in the upper half of the diagram (area 474) and tissue affected by −2.56 V shown on the lower half of the diagram (area 475). From this, it may be concluded that application of electrical stimulus to tissue sites of the upper electrode and the lower electrode result in electrical stimulus to a relatively large area of tissue not associated with the tissue sites of the electrodes, i.e., a relatively large area of tissue in which electrical stimulus is preferably to be avoided.

FIG. 7 illustrates a first shielded, bipolar, biphasic pacing modeled result associated with variable pacing stimulation devices, systems, and methods. As can be seen, when 0 V is applied on upper electrode 402k, and 5 V is applied on the lower electrode 4021, and 0 voltage is applied on the ring 404 of electrodes surrounding the upper electrode and the lower electrode, e.g., electrodes 402a-402j in FIG. 4, tissue relatively far away from the tissue site of the upper electrode and the lower electrode is not affected by voltage, e.g., the result is tissue receiving 0 V shown in the upper half of the diagram (area 476) and tissue receiving 0 V shown on the lower half of the diagram (area 477). From this, it may be concluded that application of electrical stimulus to tissue sites of the upper electrode and the lower electrode while 0V applied to a ring of electrodes surrounding the upper electrode and the lower electrode result in virtually no electrical stimulus to a relatively large area of tissue not associated with the tissue sites of the electrodes, i.e., the target tissue sites receive desired amounts of electrical stimulus while the electrical stimulus of non-targeted tissues sites (relatively large area of tissue surrounding the target sites) is avoided.

FIG. 7 provides terminology permitting a description of a second shielded, bipolar, biphasic pacing modeled result associated with variable pacing stimulation devices, systems, and methods. When 0 V is applied on upper electrode 402k, and −5 V is applied on the lower electrode 4021, and 0 V is applied on the ring 404 of electrodes surrounding electrodes 402a-402j, the modeled result is that tissue relatively far away from the tissue site of the upper electrode and the lower electrode is not affected by voltage, e.g., tissue receives 0 V in the upper half of the diagram and tissue receives 0 V in the lower half of the diagram. From this, it may be concluded that application of electrical stimulus to tissue sites of the upper electrode and the lower electrode while 0 V is applied to a ring of electrodes surrounding the upper electrode and the lower electrode result in virtually no electrical stimulus to a relatively large area of tissue not associated with the tissue sites of the electrodes, i.e., the target tissue sites receive desired amounts of electrical stimulus while the electrical stimulus of non-targeted tissues sites (relatively large area of tissue surrounding the target sites) is avoided.

Similar modeling of the case of −2.5V on the upper electrode, and 2.5V on the lower electrode, and 0V on the shield electrodes, yields very nearly identical results on distant tissue, namely a modeled result of about 0V on the distant tissue for example in areas 476 and 477.

Similar modeling of the case of 2.5V on the upper electrode, and −2.5V on the lower electrode, and 0V on the shield electrodes, yields very nearly identical results on distant tissue, namely a modeled result of about 0V on the distant tissue for example in areas 476 and 477.

Returning now to the subject of things which may vary in the stimulation techniques, various combinations of variability parameters are possible. One example of such a combination includes a focus variability parameter and a voltage variability parameter. One example of an implementation of the illustrative combination is provided in a configuration having a ring of electrodes shielding an upper electrode and a lower electrode to focus stimulation to a targeted tissue site while ensuring focus of the stimulation away from untargeted sites (as previously described with respect to the focus variability parameter) coupled with an increase in the intensity of the stimulation at the targeted site, e.g., via the voltage variability parameter (as previously described with respect to the voltage variability parameter). In this manner, the beneficial gain realized by intensifying the stimulation of the targeted site may only be realized when the non-targeted surrounding tissue areas are protected from the intensified levels of voltage.

An aspect of the invention includes steps programmably controlling one or more variability parameters with one or more lead integrated circuits; and generating electrical stimulation based on the one or more variability parameters via one or more electrodes, each electrode individually addressable by at least one of the lead integrated circuits.

Another aspect of the invention comprises one or more lead integrated circuits, wherein each lead integrated circuit has a programming module to programmably control one or more variability parameters; and one or more electrodes, each electrode individually addressable by at least one of the lead integrated circuits and controlled by at least one variability parameter.

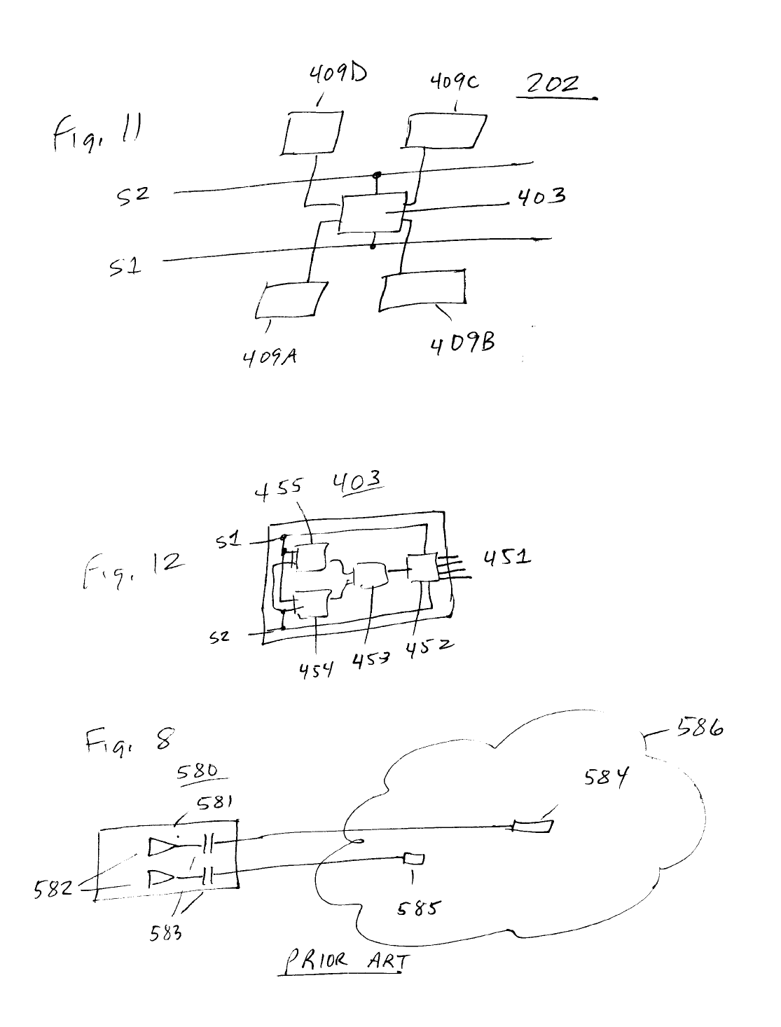

It will be helpful to review one prior-art way of driving electrodes, shown schematically in FIG. 8. In this approach, a system 580 has a “can” 581 with drivers 582 coupled by means of capacitors 583. The capacitors 583 are chosen to substantially block DC flows to electrodes 584, 585 and to pass only signals that are time-variant, such as AC signals or pulses of DC.

One insight that has helped to develop the embodiment of the invention now being discussed is the realization that tissue 586 that is in contact with electrodes 584, 585 may be modeled in a variety of ways, and one model that turns out to have some success is a model that assumes a diode-like behavior, or perhaps more generally a nonlinear behavior. On this model, the system (which includes capacitors 583 as well as the modeled behavior of the tissue 586) can maintain a non-negligible stored potential in the neighborhood of electrodes 584, 585. The practical prediction of this model, a prediction that seems to be borne out in some actual results, is degradation (corrosion) of the electrodes 584, 585 sooner than might otherwise occur.

In one embodiment of the invention, a “shorting” technique is employed to dissipate any such non-negligible stored potential as might have developed due to pacing or stimulation. FIG. 9 shows a system 592 employing the “shorting” technique.

Can 591 may be seen. The can 591 might contain capacitively coupled drives such as those shown in can 581 in FIG. 8, or might contain other types of drivers, without departing from this aspect of the invention. Lead 593 is shown with two satellites each with a respective chip 594, 595. (The number of satellites might be greater than two.) Each chip is shown with four electrodes including electrodes 596, 597. (The number of electrodes could be different without departing from the invention.) In an exemplary sequence of events, control signals pass from can 591 along lead 593 to instruct chip 594 to connect line 599 with electrode 596. Other control signals pass from can 591 along lead 593 to instruct chip 595 to connect line 598 with electrode 597. Then a pacing pulse or stimulation signal is emitted by can 591 and passes to electrodes 596 and 597. The signal might be AC or might be DC. It might be a pulse in which one electrode is driven negative relative to the other, and is later drive positive relative to the other, in an effort to approximate a charge balance.

In any of these cases of types of pacing or stimulation, the concern is that when the pacing or stimulation is done, a residual potential might remain in the system. According to the invention, then, what happens for example is that a control signal is sent from can 591 to chip 594 to cause it to connect electrode 596 to line 598. Another control signal is sent from can 591 to chip 595 to cause it to connect electrode 597 to line 598. (It will be appreciated that perhaps chip 595 will already be connecting these elements together from the previous steps.)

The practical consequence of this action is that electrodes 596 and 597 are shorted together. The goal is to dissipate some or all of any residual potential that might have remained in the system. This should help to extend the life of the electrodes 596 and 597.

This sequence of steps is described with two electrodes 596 and 597 but it will be appreciated that it may be advantageous to short together all of the electrodes along the lead.

While there are many applications where a two-wire lead 593 is thought to be optimal, the teachings of the invention offer their benefits even in a one-wire system. Consider for example FIG. 10 which shows system 601. This system is characterized by a lead with only one wire, 608 in which the “return path” for control signals as well as other current flows is through the tissue to separate electrode 605. This may be another lead just like the one with chip 606 or may be a simple coil of wire 605. When the stimulation is done, chip 606 shorts the electrode or electrodes of interest (such as electrode 604) to the line 608. Switch 603 shorts line 608 to line 605. In this way, residual potential may be dissipated.

Consider also FIG. 13 which shows system 611. This system, like that of system 601 (FIG. 10) is characterized by a lead with only one wire 618, in which the “return path” for control signals as well as other current flows is through the tissue to the housing 617 of the can 612. When the stimulation is done, chip 616 shorts the electrode or electrodes of interest (such as electrode 614) to the line 618. Switch 613 shorts line 618 to housing 617. In this way, residual potential may be dissipated.

Any of several duty cycles may be followed depending on other constraints or needs (for example depending on the organ or system of the body being stimulated or the nature of the condition being treated). One approach would be to carry out the stimulation once, and then to leave many or most electrodes shorted thereafter until just before the start of the next stimulation. A different approach is to carry out the stimulation once, and then to short out many or most electrodes briefly, thereafter letting the electrodes “float” until just before the start of the next stimulation.

Such a “floating” time may be helpful so as to facilitate data-gathering such as sensing conditions in the tissue of interest.

It is also contemplated that instead of a direct short, a fixed impedence of around half of a megohm might be left in place at all times. Such an impedence would not interfere with pacing or stimulation pulses, and would not interfere with data gathering between pulses, and yet might permit some dissipation of residual potentials. This impedence might straddle some or all of the switches that make up the switching fabric discussed above.

One or more aspects of the subject invention may be in the form of computer readable media having programming stored thereon for implementing the various methods, or various steps thereof. The computer readable media may be, for example, in the form of a computer disk or CD, a floppy disc, a magnetic “hard card”, a server, or any other computer readable media capable of containing data or the like, stored electronically, magnetically, optically or by other means. Accordingly, stored programming embodying steps for carrying out the subject methods may be transferred or communicated to a processor, e.g., by using a computer network, server, or other interface connection, e.g., the Internet, or other relay means. It is to be understood that this invention is not limited to particular aspects described, and, as such, may vary. It is also to be understood that the terminology used herein is for the purpose of describing particular aspects only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference in their entirety as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent upon reading this disclosure, each of the individual aspects described and illustrated herein has discrete components and features which may be readily separated from or combined with the features of any of the other several aspects without departing from the scope or spirit of the present invention. Any recited method can be carried out in the order of events recited or in any other order which is logically possible.

Although the foregoing invention has been described in some detail by way of illustration and example for purposes of clarity of understanding, it is readily apparent in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and aspects of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary aspects shown and described herein. Rather, the scope and spirit of present invention is embodied by the appended claims.

1. A device, comprising:
one or more lead integrated circuits to programmably control one or more variability parameters; and
one or more electrodes to provide electrical stimulation, each electrode individually addressable by at least one of the lead integrated circuits and controlled by at least one variability parameter.

2. The device of claim 1, wherein the variability parameters is selected from a group consisting essentially of a voltage variability parameter; a spread spectrum variability parameter; a pacing variability parameter; a delay variability parameter; a frequency variability parameter; an interval variability parameter; an amplitude variability parameter; a component variability parameter; a state variability parameter; a blocking variability parameter; a potential variability parameter; and a focus variability parameter.

3. The device of claim 2, wherein the voltage variability parameter comprises varying a voltage associated with one or more electrodes.

4. The device of claim 2, wherein the pacing variability parameter comprises pseudo-randomly varying a pace of electrical stimuli.

5. The device of claim 2, wherein the state variability parameter comprises selectively activating or deactivating one or more electrodes.

6. The device of claim 2, wherein the component variability parameter comprises individual addressability of the one or more electrodes by the lead integrated circuit.

7. The device of claim 6, wherein the electrodes are configured as segmented electrode satellites.

8. The device of claim 7, wherein the segmented electrode satellites comprise at least one of a four-segmented electrode and a six-segmented electrode.

9. The device of claim 6, wherein the electrodes are configured in an array of electrodes.

10. The device of claim 7, wherein the array of electrodes comprises an array of arrays.

11. The device of claim 2, wherein the focus variability parameter comprises alternating between voltages that are positive or negative associated with a first group of electrodes while maintaining a neutral voltage associated with a second group of electrodes.

12. The device of claim 1, wherein at least one of the one or more variability parameters is generated via a scheme selected from a group consisting of a random generation scheme and a pseudo-random generation scheme.

13. The device of claim 1, further comprising:
a bus coupled to the one or more lead integrated circuits; and
a power supply associated with the bus.

14. A method, comprising:
programmably controlling one or more variability parameters with one or more lead integrated circuits; and
generating electrical stimulation based on the one or more variability parameters via one or more electrodes, each electrode individually addressable by at least one of the lead integrated circuits.

15. The method of claim 14, wherein the variability parameters is selected from a group consisting essentially of a voltage variability parameter; a spread spectrum variability parameter; a pacing variability parameter; a delay variability parameter; a frequency variability parameter; an interval variability parameter; an amplitude variability parameter; a component variability parameter; a state variability parameter; a blocking variability parameter; a potential variability parameter; and a focus variability parameter.

16. The method of claim 15, wherein the voltage variability parameter comprises varying a voltage associated with one more electrodes.

17. The method of claim 15, wherein the pacing variability parameter comprises pseudo-randomly varying a pace of electrical stimuli.

18. The method of claim 15, wherein the state variability parameter comprises selectively activating or deactivating one or more electrodes.

19. The method of claim 15, wherein the component variability parameter comprises individual addressability of the one or more electrodes by the lead integrated circuit.

20. The method of claim 19, wherein the electrodes are configured as segmented electrode satellites.

21. The method of claim 20, wherein the segmented electrode satellites comprise at least one of a four-segmented electrode and a six-segmented electrode.

22. The method of claim 19, wherein the electrodes are configured in an array of electrodes.

23. The method of claim 20, wherein the array of electrodes comprises an array of arrays.

24. The method of claim 15, wherein the focus variability parameter comprises alternating between voltages that are positive or negative associated with a first group of electrodes while maintaining a neutral voltage associated with a second group of electrodes.

25. The method of claim 14, wherein at least one of the one or more variability parameters is generated via a scheme selected from a group consisting of a random generation scheme and a pseudo-random generation scheme.

26. A system comprising:
one or more lead integrated circuits, each lead integrated circuit having:
a programming module to programmably control one or more variability parameters; and one or more electrodes, each electrode individually addressable by at least one of the lead integrated circuits and controlled by at least one variability parameter.

27. A method for use with a lead having at least first and second satellites along its length, each satellite having at least two electrodes communicatively coupled thereto, the lead having at least one conductor coupled to the satellites, the total number of electrodes coupled with the satellites along the lead exceeding the number of conductors in the lead, the method comprising the steps of:
by means of control signals transmitted along the lead to the satellites, closing solid-state switches in the satellites, causing at least one electrode at the at least first satellite to be shorted to the at least one conductor, and at the same time, causing at least one electrode at the at least second satellite to be shorted to the at least one conductor, whereby the at least one electrode at the at least first satellite is shorted to the at least one electrode at the at least second satellite;
ceasing the shorting of the at least one electrode at the at least first satellite to the at least one electrode at the at least second satellite;
passing a stimulus signal along the lead to a selected electrode;
ceasing the passing of a stimulus signal along the lead to the selected electrode;
by means of control signals transmitted along the lead to the satellites, closing solid-state switches in the satellites, causing at least one electrode at the at least first satellite to be shorted to the at least one conductor, and at the same time, causing at least one electrode at the at least second satellite to be shorted to the at least one conductor, whereby the at least one electrode at the at least first satellite is shorted to the at least one electrode at the at least second satellite; and
ceasing the shorting of the at least one electrode at the at least first satellite to the at least one electrode at the at least second satellite.

28. The method of claim 27 wherein the selected electrode is the same as the at least one electrode of the at least first satellite.

29. A method for use with a lead having at least first and second satellites along its length, each satellite having at least two electrodes communicatively coupled thereto, the lead having at least one conductor coupled to the satellites, the total number of electrodes coupled with the satellites along the lead exceeding the number of conductors in the lead, the method comprising the steps of:
by means of control signals transmitted along the lead to the satellites, closing solid-state switches in the satellites, causing a first at least one electrode at the at least first satellite to be shorted to a second at least one electrode at the at least first satellite;
ceasing the shorting of the first at least one electrode at the at least first satellite to be shorted to the second at least one electrode at the at least first satellite;
passing a stimulus signal along the lead to a selected electrode;
ceasing the passing of a stimulus signal along the lead to the selected electrode;
by means of control signals transmitted along the lead to the satellites, closing solid-state switches in the satellites, causing a first at least one electrode at the at least first satellite to be shorted to a second at least one electrode at the at least first satellite; and
ceasing the shorting of the first at least one electrode at the at least first satellite to be shorted to the second at least one electrode at the at least first satellite.

30. The method of claim 29 wherein the selected electrode is the same as the first at least one electrode of the at least first satellite.

31. The method of claim 27 or claim 29 wherein the lead has two conductors along its length.

32. A method for use with a driver,
the driver connected with a wire and a lead;
the lead having at least first and second satellites along its length, each satellite having at least two electrodes communicatively coupled thereto, the lead having at least one conductor coupled to the satellites, the total number of electrodes coupled with the satellites along the lead exceeding the number of conductors in the lead, the method comprising the steps of:
by means of control signals transmitted along the lead to the satellites, closing a solid-state switch in a first satellite, causing at least one electrode at the first satellite to be shorted to the at least one conductor, and at the same time, by means of a switch within the driver, the wire to be shorted to the at least one conductor, whereby the at least one electrode at the first satellite is shorted to the wire;
ceasing the shorting of the at least one electrode at the first satellite to the wire;
passing a stimulus signal along the lead to a selected electrode;
ceasing the passing of a stimulus signal along the lead to the selected electrode;
by means of control signals transmitted along the lead to the satellites, closing a solid-state switch in the first satellite, causing at least one electrode at the first satellite to be shorted to the at least one conductor, and at the same time, by means of a switch within the driver, causing the wire to be shorted to the at least one conductor, whereby the at least one electrode at the first satellite is shorted to the wire; and
ceasing the shorting of the at least one electrode at the first satellite to the wire.

33. The method of claim 32 wherein the lead has two conductors along its length.

34. The method of claim 32 wherein the wire and at least one electrode are in contact with tissue.

35. A method for use with a driver,
the driver having a conductive housing and connected to a lead;
the lead having at least first and second satellites along its length, each satellite having at least two electrodes communicatively coupled thereto, the lead having at least one conductor coupled to the satellites, the total number of electrodes coupled with the satellites along the lead exceeding the number of conductors in the lead, the method comprising the steps of:
by means of control signals transmitted along the lead to the satellites, closing a solid-state switch in a first satellite, causing at least one electrode at the first satellite to be shorted to the at least one conductor, and at the same time, by means of a switch within the driver, causing the housing to be shorted to the at least one conductor, whereby the at least one electrode at the first satellite is shorted to the housing;
ceasing the shorting of the at least one electrode at the first satellite to the housing;
passing a stimulus signal along the lead to a selected electrode;
ceasing the passing of a stimulus signal along the lead to the selected electrode;
by means of control signals transmitted along the lead to the satellites, closing a solid-state switch in the first satellite, causing at least one electrode at the first satellite to be shorted to the at least one conductor, and at the same time, by means of a switch within the driver, causing the housing to be shorted to the at least one conductor, whereby the at least one electrode at the first satellite is shorted to the housing; and
ceasing the shorting of the at least one electrode at the first satellite to the housing.

36. The method of claim 35 wherein the lead has two conductors along its length.

37. The method of claim 35 wherein the housing and at least one electrode are in contact with tissue.

38. A system comprising a driver,
the driver connectable with a lead and with a wire at respective connection points;
the lead having at least one conductor along its length, and having at least one chip along its length connected to the conductor, the chip connected to at least two electrodes, the number of electrodes exceeding the number of conductors along the length of the lead;
the chip comprising switches controllably and selectively connecting respective electrodes to the at least one conductor of the lead;
the driver comprising a switch controllably and selectively connecting the connection point of the wire to the connection point of the at least one conductor of the lead;
the driver comprising means for controlling the switches of the chip so as to connect at least one of the electrodes to the at least one conductor of the lead, while simultaneously controlling the switch of the driver so as to connect the connection point of the wire to the connection point of the at least one conductor of the lead.

39. The system of claim 38 wherein the lead is in sterile packaging.

40. The system of claim 39 wherein the driver is in sterile packaging.

41. A system comprising a driver,
the driver connectable with a lead at a connection point and having a conductive housing;
the lead having at least one conductor along its length, and having at least one chip along its length connected to the conductor, the chip connected to at least two electrodes, the number of electrodes exceeding the number of conductors along the length of the lead;
the chip comprising switches controllably and selectively connecting respective electrodes to the at least one conductor of the lead;
the driver comprising a switch controllably and selectively connecting the conductive housing to the connection point of the at least one conductor of the lead;
the driver comprising means for controlling the switches of the chip so as to connect at least one of the electrodes to the at least one conductor of the lead, while simultaneously controlling the switch of the driver so as to connect the conductive housing to the connection point of the at least one conductor of the lead.

42. The system of claim 41 wherein the lead is in sterile packaging.

43. The system of claim 42 wherein the driver is in sterile packaging.

44. A driver for use with a lead and with a wire, the lead of a type having at least one conductor and the lead receiving control signals by which the lead selectively connects the at least one conductor with an electrode in response to control signals;
the driver connectable with the lead and with the wire at respective connection points;
the driver comprising a switch controllably and selectively connecting the connection point of the wire to the connection point of the at least one conductor of the lead;
the driver comprising means for sending control signals disposed to control the lead so as to connect the electrode to the at least one conductor of the lead, while simultaneously controlling the switch of the driver so as to connect the connection point of the wire to the connection point of the at least one conductor of the lead.

45. The system of claim 44 wherein the driver is in sterile packaging.

46. A driver for use with a lead, the driver having a conductive housing, the lead of a type having at least one conductor and the lead receiving control signals by which the lead selectively connects the at least one conductor with an electrode in response to control signals;
the driver connectable with the lead at respective connection points;
the driver comprising a switch controllably and selectively connecting the conductive housing to the connection point of the at least one conductor of the lead;
the driver comprising means for sending control signals disposed to control the lead so as to connect the electrode to the at least one conductor of the lead, while simultaneously controlling the switch of the driver so as to connect the conductive housing to the connection point of the at least one conductor of the lead.

47. The system of claim 46 wherein the driver is in sterile packaging.
